(19) United States
(12) Patent Application Publication
(10) Pub. No.: US 2005/0124526 A1
(43) Pub. Date: Jun. 9, 2005

(54) Branched sulfates with improved odor properties and their use in personal care compositions

(76) Inventors: Paul F. D'Angelo, Princeton, NJ (US); Euen Gunn, Trenton, NJ (US)

Correspondence Address: KEVIN E. MC VEIGH;RHODIA INC., 259 PROSPECT PLAINS ROAD, CRANBURY, NJ, 08512, US

(21) Appl. No.: 11/004,375

(22) Filed: Dec. 3, 2004

Related U.S. Application Data
(60) Provisional application No. 60/526,624, filed on Dec. 3, 2003.

(51) Int. Cl. A61K 7/075

(52) U.S. Cl. 510/424

(57) ABSTRACT

Branched alkyl (ether) sulfates according to formula (I): 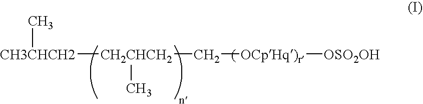 and salts thereof, wherein n′ is 3, 4 or 5, p′ is 2 or 3, q′ is 2p′, and r is 0 to 50, exhibit improved odor properties.

### FIELD OF THE INVENTION

This invention relates to branched sulfates having improved odor properties and to their use in personal care compositions.

### BACKGROUND OF THE INVENTION

Commercially available branched alkyl (ether) sulfates, that is, branched alkyl and alkyl ether sulfates, typically exhibit a very noticeable, pungent odor. This pungent odor is offensive to formulators and manufacturers of personal care products and the odoriferous residue of the sulfate can interfere with fragrances during formulating and can be retained on the hair and skin despite the use of masking fragrances and perfumes in such personal care products. This odor problem has resulted in limited use of such branched alkyl (ether) sulfates in personal care products, that is, these branched alkyl (ether) sulfates have been largely confined to use in personal care compositions, such as baby shampoos, wherein the offensive odor can be effectively masked.

Branched ether sulfates have been found to provide protection against viscosity losses in structured surfactant systems, see for example, U.S. Pat. Nos. 5,952,286, 5,962,395,6,077,816, 6,174,846, and 6,150,312, which disclose the use of branched and linear organic compounds and branched ether sulfates in such systems. However, the odor associated with branched alkyl (ether) sulfates remains an obstacle to their use in personal care applications and the commercial use of branched sulfates in such applications is likely to remain limited until this odor concern can be diminished or eliminated.

### SUMMARY OF THE INVENTION

In a first aspect, the present invention is directed to a branched alkyl (ether) sulfate, comprising one or more compounds according to formula (1): 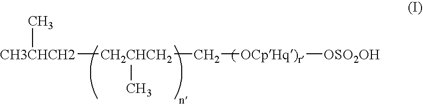
or a salt thereof, wherein
n′ is 3, 4 or 5,
p′ is 2 or 3,
q′ is 2p′, and
r is 0 to 50.

In second aspect, the present invention is directed to a branched alkyl (ether) sulfate, comprising one or more compounds, or salts thereof, made by sulfating a branched alcohol or by ethoxylating the branched alcohol and then sulfating the ethoxylated branched alcohol, wherein the branched alcohol is made by contacting one or more internal olefins with synthesis gas in the presence of a hydroformylation/hydrogenation catalyst to produce the branched alcohol in a single step.

In third aspect, the present invention is directed to a branched alkyl (ether) sulfate, comprising one or more branched alkyl (ether) sulfate compounds, or salts thereof, wherein the branched alkyl (ether) sulfate is free of aldehyde residues.

The branched alkyl (ether) sulfates of the present invention exhibit improved odor properties.

In a third aspect, the present invention is directed to a personal care composition comprising a branched alkyl (ether) sulfate of the present invention or salt thereof.

The personal care composition of the present invention exhibits improved odor properties.

### DETAILED DESCRIPTION OF THE INVENTION

Suitable branched alkyl (ether) sulfate salts include, for example, sodium, potassium and ammonium salts.

We have found that by the proper selection of the starting branched alcohol or branched alcohol ethoxylate that would be used to make the alkyl (ether) sulfates, we can largely, if not completely, eliminate the offensive odor of such branched alkyl (ether) sulfates.

Linear alcohols of C₁₂-C₁₄ carbon chains typically have a light or mild, fruity or fatty odor. It is surprising to us that branched alkyl alcohols and alcohol ethoxylates would not have comparable odor properties. For example a widely used, branched tridecyl alcohol (Exxal 13, Exxon/Mobil) from has a noticeable, pungent odor. An ethoxylate made from that alcohol (Rhodasurf BC-420, Rhodia Inc.), also has a pungent, offensive odor, as does a branched trideceth(3) sulfate made from that ethoxylate. Other commercially produced branched sulfates, such as tridecyl and trideceth(3) sulfates, exhibit pungent odors.

We found branched alcohol ethoxylates, for example, Alfonic TDA-3 ethoxylate (Sasol), which have an odor profile that is more mild and fruity compared to the Exxon/Mobil alcohols and Rhodasurf BC-420 TDA-3 ethoxylate. The perceived differences in odor properties are apparently due to differences in the manufacturing processes to make such branched alcohols and branched alcohol ethoxylates.

Synthetic branched alcohols are based on petrochemical raw materials and are typically methyl branched. The processes most commonly used to make branched alcohols are believed to be the oxo- and modified oxo-hydroformylation/hydrogenation processes.

In the oxo process, internal and/or α-olefins are contacted with synthesis gas (CO, H₂) in the presence of suitable catalyst at high temperatures and pressures to produce a mixture of linear and branched aldehydes. The aldehydes are then hydrogenated in a second step by contacting the aldehydes with hydrogen gas in the presence of a hydrogenation catalyst to make a mixture of linear and branched alcohols.

We believe that:
Exxal 13 branched tridecyl alcohol is made from propene by the oxo process and comprises from 40 to 60% branched alcohols according to formula (II): 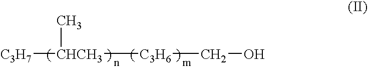
   and from 40 to 60% linear alcohols,
the Rhodia BC-420 ethoxylated branched alcohol made from the Exxal 13 branched alcohol comprises from 40 to 60% branched alcohols according to formula (III): 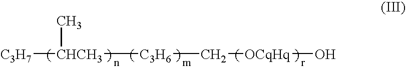
   and from 40 to 60% ethoxylated linear alcohols, and
the branched alkyl ether sulfate made from Rhodia BC-420 ethoxylated branched alcohol comprises from 40 to 60% branched alcohols according to formula (IV): 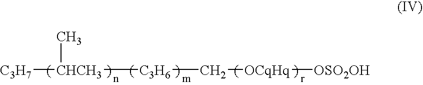
   and from 40 to 60% linear alkyl ether sulfates,
   wherein, in each case:
m+n is 3,
p is 2 or 3,
q is 2p,
r is greater than 0 to about 50, and
the branched and non-branched units are randomly distributed along the carbon chain.

In the modified oxo process, internal olefins are contacted with synthesis gas in the presence of a hydroformylation/hydrogenation catalyst, typically a cobalt phosphine ligand catalyst, to produce branched alcohols or a mixture of linear and branched alcohols by hydroformylation of the olefin to form one or more aIdehyde intermediates and hydrogenation of such aldehyde intermediates in a single step.

We believe that the alcohol from which the Alfonic TDA-3 ethoxylate is made is made from 2-butene by a modified oxo-process and comprises predominately branched alcohols according to formula (V): 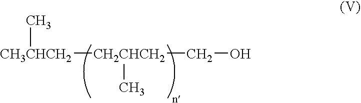
wherein n′ is 3, and that the Alfonic TDA-3 ethoxylate comprises predominately branched alcohols according to formula (VI): 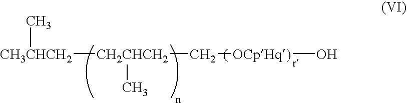
wherein n′ is 3, p′ is 2, q′ is 4, and r′ is 3.

The branched alkyl (ether) sulfate of the present invention exhibit improved odor properties compared to analogous branched alkyl (ether) sulfates, that is, branched alkyl (ether) sulfates according to formula (I) exhibit reduced odor compared to the pungent odor exhibited by branched alkyl (ether) sulfates according to formula (IV), or alternatively, branched alkyl (ether) sulfates derived from alcohols made by the modified oxo process exhibit reduced odor compared to the pungent odor exhibited by branched alkyl (ether) sulfates derived from alcohols made by oxo process.

Aldehydes have pungent, irritating odors and even small amounts could account for the odor profile differences rather than having different alcohol ethoxylate isomers with different odor profiles. The presence of aldehydes can be detected by gas chromatography. It appears that alcohols, such as Exxal 13, made by the oxo process, as well as alkoxylates of such alcohols, contain small amounts of residual unreacted aldehyde and that alcohols made by the modified oxo synthetic route, such as the alcohol precursor of Alfonic TDA-3 ethoxylate, and alkoxylates of such alcohols do not contain aldehydes.

In one embodiment, the personal care composition is a baby shampoo composition. In one embodiment, the baby shampoo composition further comprises water and PEG sorbitan laurate.

In one embodiment, the personal care composition of the present invention is an aqueous structured surfactant that comprises water and one or more anionic surfactants, exhibits shear-thinning viscosity, and is capable of suspending water insoluble or partially water soluble components.

Shear-thinning viscosity is measured by known viscometric methods, such as for example, using a rotational viscometer, such as a Brookfield viscometer. In one embodiment, the composition of the present invention exhibits shear-thinning behavior when subjected to viscosity measurement using a Brookfield rotational viscometer, equipped with an appropriate spindle, at a rotation speed of from about 0.1 revolutions per minute (“rpm”) to about 60 rpm.

The composition of the present invention is capable of suspending water-insoluble particles or partially water soluble components, such as vegetable oils, mineral oils, silicone oils, solid particles, abrasives, and similar articles. The composition provides a means to include otherwise difficult to incorporate components in surfactant mixtures resulting in cosmetic preparations with multi-functional benefits including, in some cases, cleansing, moisturizing, improved skin feel, exfoliation/abrasion, novel appearance, or a combination of these benefits.

The ability of a composition to suspend water insoluble or partially water insoluble components is typically evaluated by mixing the composition with sufficient vigor to entrap air bubbles in the composition and then visually observing whether the air bubbles remain entrapped in the composition for a defined period of time, such as for example, 12 to 24 hours, under defined environmental conditions, such as for example, room temperature. In one embodiment, the composition of the present invention is capable of suspending air bubbles for at least 1 week, and more typically for at least 3 months. A composition that is capable of suspending air bubbles under the for at least 12 hours at room temperature is deemed to be generally capable of suspending water insoluble or partially water soluble components in the composition under generally anticipated processing, storage, and use conditions for such composition. For components other than air, the result of the air suspension test should be confirmed by conducting an analogous suspension test using the component of interest. For unusually rigorous processing, storage and/or use conditions, more rigorous testing may be appropriate.

In one embodiment, the ability to suspend water insoluble or partially water insoluble components is evaluated under more rigorous conditions, that is, the mixed samples are visually evaluated after subjecting the samples to one or more freeze/thaw cycles, wherein each freeze/thaw cycle consists of 12 hours at −10° C. and 12 hours at 25° C. In one embodiment, composition of the present invention remains capable of suspending air bubbles after one freeze/thaw cycle, more typically after 3 freeze/thaw cycles.

In one embodiment, the structured surfactant composition of the present invention comprises from about 3 to about 40 pbw, more typically from about 5 to about 30 pbw, and still more typically from about 8 to about 20 pbw, of the one or more anionic surfactants. Suitable anionic surfactants are known in the art.

In one embodiment, the structured surfactant composition of the present invention further comprises at least an effective amount of one or more structuring agents. Suitable structuring agents are known compounds and include cationic surfactants, fatty alcohols, alkoxylated alcohols, fatty acids, fatty acid esters, alkanolamides, and electrolytes. An effective amount of such structuring agent is one that can aid in the formation of a shear-thinning phase capable of suspending water insoluble or partially water soluble components.

The composition of the present invention may optionally further comprise, in addition to the anionic surfactant and any structuring agent, one or more cationic surfactants, one or more non-ionic surfactants, one or more electrolytes, one or more amphoteric surfactants, one or more zwitterionic surfactants, or a mixture thereof. In cases where such optional components may function as a structurant, each of such components may independently be present in an amount in excess of the minimum amount effective to act as a structurant. Suitable cationic surfactants, non-ionic surfactants, electrolytes, amphoteric surfactants, and zwitterionic surfactants are known in the art.

In one embodiment, the personal care composition of the present invention further comprises one or more benefit agents, such as emollients, moisturizers, conditioners, skin conditioners, hair conditioners, vitamins or their derivatives, antioxidants, free-radical scavengers, abrasives, dyes, hair coloring agents, bleaching agents, hair bleaching agents, anti-UV agents, UV absorbers, antimicrobial agents, antibacterial agents, antifungal agents, melanin regulators, tanning accelerators, depigmenting agents, skin-coloring agents, liporegulators, weight-reduction agents, anti-acne agents, antiseborrhoeic agents, anti-ageing agents, anti-wrinkle agents, keratolytic agents, anti-inflammatory agents, refreshing agents, cicatrizing agents, vascular-protection agents, antiperspirants, deodorants, immunomodulators, nourishing agents, agents for combating hair loss, reducing agents for permanent-waving, essential oils and fragrances.

In one embodiment, the personal care composition of the present invention further comprises one or more benefit agents, such as emollients, moisturizers, conditioners, skin conditioners, hair conditioners, vitamins or their derivatives, antioxidants, free-radical scavengers, abrasives, dyes, hair coloring agents, bleaching agents, hair bleaching agents, anti-UV agents, UV absorbers, antimicrobial agents, antibacterial agents, antifungal agents, melanin regulators, tanning accelerators, depigmenting agents, skin-coloring agents, liporegulators, weight-reduction agents, anti-acne agents, antiseborrhoeic agents, anti-ageing agents, anti-wrinkle agents, keratolytic agents, anti-inflammatory agents, refreshing agents, cicatrizing agents, vascular-protection agents, antiperspirants, deodorants, immunomodulators, nourishing agents, agents for combating hair loss, reducing agents for permanent-waving, essential oils and fragrances.

The personal care composition of the present invention exhibits improved odor properties, that is, reduced odor, compared to the pungent odor exhibited by analogous personal care compositions that contain branched alkyl (ether) sulfates according to formula (IV), or alternatively, branched alkyl (ether) sulfates derived from alcohols made by oxo process.

### EXAMPLE 1

The sodium trideceth(3) sulfate composition of Example 1 (30% active in deionized water) was made by sulfating Alfonic TDA-3 branched alkyl 3 mole ethoxylate (Sasol) by contacting the ethoxylate with SO₃ in a falling film evaporator and forming its sodium salt by neutralizing the sulfate with sodium hydroxide.

Rhodapex EST-30 (Rhodia Inc.) was used as the sodium trideceth(3) sulfate composition of Comparative Example C1 (30% active in deionized water). The composition of Comparative Example C1 was made by sulfating Rhodasurf BC-420 TDA-3 exthoxylate (Rhodia Inc.) and forming its sodium salt. Rhodasurf BC-420 TDA-3 exthoxylate was made by ethoxylating Exxal 13 tridecyl alcohol (Exxon/Mobil).

The sodium trideceth(3) sulfate composition of Example 1 exhibited a mild odor with a light, fruity quality while (sodium trideceth(3) sulfate composition of Comparative Example C1 exhibited a relatively harsh, sharp, pungent odor.

### EXAMPLE 2

The body wash of Example 2 was prepared using the branched alkyl ether sulfate of Example 1 and compared with the analogous body wash of Comparative Example C2, which was made using a branched alkyl ether sulfate derived from the 3 mole ethoxylate of Exxal 13 branched C₁₃ alcohol.

The compositions of Example 2 and Comparative Example C2 were each made as follows. Into main vessel was charged a surfactant blend (Miranol L-32 (Rhodia)), the branched alkyl ether sulfate and deionized water. The mixture was mixed and heated to a temperature in the range of 65° C. to 70° C. Coco mono-ethanol amide (CMEA) was charged to a separate vessel and heated to 70° C. Once the mixture and CMEA each reached 65° C. to 70° C., molten CMEA was charged into main vessel and mixed at temperature for 30 minutes. The heating was then discontinued while maintaining mixing, adding citric acid and sodium chloride.

Composition of Example 2 (1350 g, pH=6.08)

| Component | wt (g) | wt % |
|---|---|---|
| Branched alkyl ether sulfate of Example 1 | 683 | 50.6 |
| Miranol Ultra L-32 (Rhodia) | 210 | 15.56 |
| CMEA (Alkamide) | 42 | 3.11 |
| Water | 363.2 | 26.9 |
| Citric Acid | 24.2 | q.s. to pH 6 |
| Sodium Chloride | 27 | 2.00 |

Composition of Comparative Example C2 (1348.7 g, 1pH=6.12)

| Component | wt (g) | wt % |
|---|---|---|
| Branched alkyl ether sulfate | 682 | 50.6 |
| (Rhodapex EST30) | | |
| Miranol Ultra L-32 (Rhodia) | 210 | 15.56 |
| CMEA (Alkamide) | 42 | 3.11 |
| Water | 362 | 26.9 |
| Citric Acid | 24.2 | q.s. to pH 6 |
| Sodium Chloride | 27 | 2.00 |

Samples of the compositions of Example 3 and Comparative Example C3 were each evaluated for odor, appearance, and stability. The smell of a composition was qualitatively evaluated by smelling a sample of the composition. The appearance of a composition was evaluated by visual inspection of a sample of the composition. The oven stability of a composition was evaluated by visual inspection of a sample of the composition after maintaining the sample in an oven at 45° C. for 3 months. The freeze/thaw stability was evaluated by visual inspection of a sample of the composition after subjecting the sample to 3 cycles of freezing and thawing the sample wherein each freeze/thaw cycle consisted subjecting the sample to −10° C. for 12 hours and then subjecting the sample to 25° C. for 12 hours. Results are set forth in Table I below.

TABLE I

| | | | Oven Stability | 3 Freeze/ |
|---|---|---|---|---|
| Ex# | Smell | Appearance | (45° C., 3 Months) | Thaw Cycles |
| 2 | none | opaque liquid | pass | pass |
| C2 | pungent | opaque liquid | pass | pass |
| | odor | | | |

### EXAMPLE 3

The baby shampoo of Example 3 was prepared using the branched alkyl ether sulfate of Example 1 and the analogous baby shampoo of Comparative Example C3, which was made using a branched alkyl ether sulfate derived from the 3 mole ethoxylate of Exxal 13 branched C₁₃ alcohol.

The compositions of Example C3 and Comparative Example C3 were each made as follows. Water was charged into the mixing vessel and heated to 65° C., with smooth agitation, the other ingredients were blended in the order listed. They were mixed until uniform. The solution was then cooled to 40° C.

Composition of Example 3

| Component | wt % |
|---|---|
| Branched alkyl ether sulfate of Example 1 | 13.1 |
| Mirataine CBS (Rhodia Inc.) | 4.4 |
| Miranol BM Conc (Rhodia Inc.) | 3.8 |
| Miranate LEC | 0.8 |
| Alkamuls PSML 80 | 6.4 |
| Alkamuls PEG 6000D | 2.0 |
| Citric Acid | Q.S. to pH 6.8 |
| Citric Acid | 69.5 |

Composition of Comparative Example C3

| Component | wt % |
|---|---|
| Rhodapex EST 30 1 | 13.1 |
| Mirataine CBS (Rhodia Inc.) | 4.4 |
| Miranol BM Conc (Rhodia Inc.) | 3.8 |
| Miranate LEC | 0.8 |
| Alkamuls PSML 80 | 6.4 |
| Alkamuls PEG 6000D | 2.0 |
| Citric Acid | Q.S. to pH 6.8 |
| Citric Acid | 69.5 |

Samples of the compositions of Example 3 and Comparative Example C3 were each evaluated for smell, appearance, % non-volatiles, viscosity, and stability. The smell, appearance, and stability of the compositions were evaluated in the manner described above in regard to the compositions of Example 2 and Comparative Example C2. The viscosity of a sample was measured using a Brookfield rotational viscometer. Results are set forth in Table II below.

TABLE II

| | | | Non | | Oven stability |
|---|---|---|---|---|---|
| | | Appearance | Volatiles | Viscosity | (45° C., |
| Ex# | Smell | (25° C.) | % | (25° C.) | 3 Months) |
| 3 | none | clear liquid | 14.3 | 1350 cps | pass |
| C3 | pungent | clear liquid | 14.4 | 1350 cps | pass |
| | odor | | | | |

1. A branched alkyl (ether) sulfate, comprising one or more compounds according to formula (I): 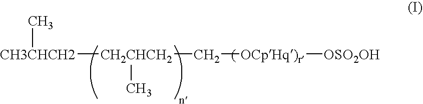
wherein
n′ is 3, 4 or 5,
p′ is 2 or 3,
q′ is 2p′, and
r is 0 to 50.

2. The branched alkyl sulfate or branched alkyl ether sulfate of claim 1, wherein n′ is 3, 4, or 5.

3. The branched alkyl (ether) sulfate of claim 1, wherein n′ is 3.

4. The branched alkyl ether sulfate of claim 1, wherein n′ is 3, p′ is 2, q′ is 4 and r′ is 3.

5. The branched alkyl (ether) sulfate of claim 1, wherein the branched alkyl (ether) sulfate is in the form of a salt.

6. The branched alkyl (ether) sulfate of claim 1, wherein the branched alkyl (ether) sulfate is in the form of a sodium salt.

7. The branched alkyl ether sulfate of claim 1, wherein the branched alkyl ether sulfate is free of aldehydes.

8. A personal care composition comprising a branched alkyl (ether) sulfate according to claim 1.

9. The personal care composition of claim 8, wherein the personal care composition is selected from shampoos, body washes, hand soaps, lotions, creams, conditioners, shaving products, facial washes, neutralizing shampoos, and skin treatments.

10. The personal care composition of claim 8, further comprising a personal care benefit agent.

11. The personal care composition of claim 8, wherein the personal care composition is a baby shampoo.

12. The personal care composition of claim 11, wherein the personal care composition further comprises water and PEG sorbitan laurate.

13. The personal care composition of claim 8, further comprising water and one or more anionic surfactants.

14. The personal care composition of claim 8, wherein the composition is an aqueous structured surfactant system that further comprises water and one or more anionic surfactants, that exhibits shear-thinning viscosity, and that is capable of suspending water insoluble or partially water soluble components.

15. A branched alkyl (ether) sulfate, comprising one or more compounds, or salts thereof, made by sulfating a branched alcohol or by ethoxylating the branched alcohol and then sulfating the ethoxylated branched alcohol, wherein the branched alcohol is made by contacting one or more internal olefins with synthesis gas in the presence of a hydroformylation/hydrogenation catalyst to produce the alcohol in a single step.

16. The branched alkyl (ether) sulfate of claim 15, wherein the branched alkyl (ether) sulfate is a branched (C₁₃)alkyl ether sulfate having an average of three moles of ethoxylation per mole of branched alkyl (ether) sulfate.

17. The branched alkyl (ether) sulfate of claim 15, wherein the branched alkyl (ether) sulfate is free of aldehydes.

18. A personal care composition comprising a branched alkyl (ether) sulfate of claim 15 or salt thereof.

19. A branched alkyl (ether) sulfate, comprising one or more branched alkyl (ether) sulfate compounds, or salts thereof, wherein the branched alkyl (ether) sulfate is free of aldehyde residues.
